(19) United States
(12) Patent Application Publication
(10) Pub. No.: US 2003/0212331 A1
(43) Pub. Date: Nov. 13, 2003

(54) Ultrasonic soft tissue cutting and coagulation systems having multiple superposed vibrational modes

(76) Inventors: Paul Fenton, Marblehead, MA (US); Francis Harrington, Peabody, MA (US); Paul Westhaver, Newburyport, MA (US)

Correspondence Address: Mark G. Lappin, McDermott, Will & Emery, 28 State Street, Boston, MA, 02109, US

(21) Appl. No.: 10/436,714

(22) Filed: May 13, 2003

Related U.S. Application Data
(60) Provisional application No. 60/380,242, filed on May 13, 2002.

(51) Int. Cl. A61B 8/14

(52) U.S. Cl. 600/459

(57) ABSTRACT

Ultrasonic soft tissue cutting or coagulating systems are featured in which multiple modes of vibration can be used simultaneously in order to vibrate an ultrasonic member, whose vibrational motion is a harmonic superposition of a plurality of modes of vibrations. Non-longitudinal modes of vibration, i.e. vibratory modes for which the direction of the vibrational motion includes at least one component that is non-parallel to the longitudinal axis of the vibrating element are excited, are stimulated. In one embodiment, a single source may excite the multiple modes of vibration that forms the composite vibratory motion. The multiple modes may include, but are not limited to, transverse, rotational, extensional, bending, and flexural modes of vibration.

### CROSS-REFERENCE TO RELATED APPLICATIONS

The present application claims priority to provisional U.S. patent application Ser. No. 60/380,242, filed on May 13, 2002, which is assigned to the assignee of the present application and incorporated herein by reference.

### STATEMENT REGARDING FEDERALLY SPONSORED RESEARCH

Not Applicable

### REFERENCE TO MICROFICHE APPENDIX

Not Applicable

### BACKGROUND OF THE INVENTION

For many years, ultrasonic surgical instruments have been used for soft tissue cutting and coagulation. These ultrasonic instruments include ultrasonic transducers which convert the electric energy supplied by a generator into ultrasonic frequency vibratory energy, which can then be applied to the tissue of a patient. Ultrasonic surgical instruments use relatively high-power, low-frequency vibratory energy, typically at a frequency range of about 20 kHz to about 100 kHz.

In general, ultrasonic soft tissue cutting and coagulation systems include a probe or horn that is coupled to the ultrasonic transducers, and thus can be made to vibrate at ultrasonic frequencies. The ultrasonically vibrating probe is then applied to the tissue, in order to transmit ultrasonic energy to the tissue. In this way, the contacted tissue can be cut or coagulated.

The mechanism through which the ultrasonic probe and the tissue interact, i.e. the physics of ultrasonic soft tissue cutting and coagulation, is not completely understood, however various explanations have been provided by researchers over the years. These explanations include descriptions of mechanical effects and thermal effects. The mechanical viewpoint states that the vibrating tip of the ultrasonic probe generates short-range forces and pressures, which are sufficient to dislodge cells in the tissue, and break up the tissue structures. Various types of forces are postulated as contributing to the rupture of the tissue layer, for example the impact forces resulting from the direct contact of the vibrating tip with tissue, and the shear forces that are the result of the differences in force levels across tissue boundaries. Some energy may be lost due to frictional heating, and by the heating caused by the absorption of acoustic energy by tissue.

Thermal effects may include frictional heat, generated by the ultrasonically vibrating tip, in an amount sufficient to melt a portion of the contacted tissue. Alternatively, the tissue may absorb the vibratory energy, which it then converts into heat. The generated heat may be used to coagulate a blood vessel, by way of example. Other effects that have been postulated in order to explain the probe-tissue interaction include cavitational effects. The cavitation viewpoint postulates that the coupling of ultrasonic energy onto tissue results in the occurrence of cavitation in tissue, namely the formation of gas or vapor-filled cavities or bubbles within the tissue, which may oscillate and propagate. A combination of mechanical, thermal, and cavitational effects may result in the desired surgical outcomes, such as cutting and coagulation.

A number of ultrasonic soft tissue cutting and coagulating systems have been disclosed in the prior art. For example, U.S. Pat. No. 5,322,055 (the “'055 patent”), entitled “Clamp Coagulator/Cutting System For Ultrasonic Surgical Instruments,” issued to T. W. Davison et al. on Jun. 21, 1994, and is assigned on its face to Ultracision, Inc. The '055 patent discloses ultrasonic surgical instruments having a non-vibrating clamp for pressing tissue against an ultrasonically vibrating blade, for cutting, coagulating, and blunt-dissecting of tissue. The '055 patent relates to ultrasonic surgical instruments having a non-vibrating clamp for pressing tissue against an ultrasonically vibrating blade, for cutting, coagulating, and blunt-dissecting of tissue. When ultrasonically activated, the blade undergoes longitudinal mode vibrations, parallel to the blade edge. The blade is used in conjunction with the clamp to apply a compressive force to the tissue in a direction normal to the longitudinal direction of vibration.

U.S. Pat. No. 6,036,667 (the “'667 patent”), entitled “Ultrasonic Dissection and Coagulation System,” issued to R. Manna et al. on Mar. 14, 2000, and is assigned on its face to United States Surgical Corporation and to Misonix Incorporated. The '667 patent discloses an ultrasonic dissection and coagulation system, including a housing, and an elongated body portion extending from the housing. The ultrasonic system includes an ultrasonic cutting blade, and a clamp member for clamping tissue in conjunction with the blade. The blade is connected, through a vibration coupler, to an ultrasonic transducer enclosed within the housing. The blade has a cutting surface that is angled with respect to the longitudinal axis of the elongated body portion.

U.S. Pat. No. 6,056,735 (the “'735 patent”), entitled “Ultrasound Treatment System,” issued to M. Okada et al. on May 2, 2000, and is assigned on its face to Olympus Optical Co., Ltd. The '735 patent relates to ultrasonic treatment systems, including endoscopic systems and aspiration systems, for treating living tissue. The '735 patent features an ultrasonic treatment system including ultrasonic transducers, and a probe that is connected to the transducers. The probe conveys ultrasonic vibrations to a stationary distal member, which forms a treatment unit together with a movable holding member. The stationary distal member and the movable holding member cooperate to clamp or free tissue between their respective surfaces, when manipulated by a scissors-like manipulating means. A turning mechanism turns the treatment unit relative to the manipulating means.

U.S. application Ser. No. ______ (filed on even date herewith and hereby incorporated by reference)(characterized by attorney docket number AXYL-185)(hereinafter the “AXYL-185 application”) discloses ultrasonic soft-tissue cutting or coagulating systems that include an ultrasonically vibrating element or blade, and a receiving clamp element, at least one of which has a substantially curvilinear configuration. The AXYL-185 application also discloses that the curvilinear configurations of the vibrating blade and/or the clamp element can be optimized, in order to improve the coupling of ultrasonic energy to the tissue being treated.

Ultrasonic blade and clamp assemblies which have curvilinear configurations can ensure a substantially uniform delivery of ultrasonic energy to the tissue that is in contact with the operative surface of the ultrasonically vibrating blade. Curvilinear configurations of the blade/clamp assemblies can also enable tissue to be treated according to a desired spatial distribution of ultrasonic energy across the contact surface. For example, the blade/clamp assemblies can be operated so that certain portions of the contacted tissue receive higher energy doses than others, for maximum surgical effect.

In the prior art ultrasonic systems described above, the vibrations of the ultrasonically vibrating element (the component which receives ultrasonic energy and transmits the ultrasonic energy to the tissue) are limited to longitudinal mode vibrations, i.e. vibrations that are parallel to a longitudinal axis of the vibrating member. In fact, some prior art patents seek to intentionally suppress transverse modes of vibration.

It is desirable to provide a multiple wavelength probe, which enables the simultaneous use of multiple modes of vibration to vibrate a distal probe.

It is also desirable to provide an ultrasonic surgical system having a vibrating element which undergoes vibrational modes that include non-longitudinal modes of vibration, for example transverse, rotational, or flexural modes of vibration, so that a wider variety of surgical effects may be achieved.

In particular, it is desirable to stimulate transverse and rotational modes of vibration, so that the vibrating element can undergo motion perpendicular to the longitudinal axis of the probe.

### SUMMARY OF THE INVENTION

The present invention is directed to ultrasonic soft tissue cutting or coagulating systems in which multiple modes of vibration can be used simultaneously in order to harmonically vibrate an ultrasonic member. The present invention is further directed to ultrasonic soft tissue cutting or coagulating systems in which the ultrasonically vibrating elements undergo non-longitudinal modes of vibration, i.e. vibratory modes for which the direction of the vibrational motion includes at least one component that is non-parallel to the longitudinal axis of the vibrating element.

An ultrasonic surgical instrument, constructed in accordance with a preferred embodiment of the present invention, includes an ultrasonic transducer for generating ultrasonic vibrations. An elongated ultrasonic coupler extends along a coupler axis. The ultrasonic coupler has a proximal end connected to the transducer to receive ultrasonic vibrations therefrom, and a distal end. The ultrasonic coupler is adapted to transmit the ultrasonic vibrations received at the proximal end to the distal end. A vibration element is connected to the distal end of the coupler for receiving ultrasonic vibrations therefrom so as to undergo vibrational motion.

In one form, the vibration element is formed of a flexible, compliant material, for example a polymer. In one embodiment of the invention, the vibration element has a substantially curvilinear configuration.

In one embodiment, the vibration element is configured so that the direction of the vibrational motion of the vibration element includes at least one component non-parallel to the longitudinal axis.

In one embodiment of the invention, the vibration element is configured so that its vibrational motion is a harmonic superposition of multiple, simultaneous modes of vibration, all of which may be excited by a single mode source.

In one embodiment, the plurality of vibratory modes of the vibration element may include, but is not limited to, transverse modes of vibration, rotational modes of vibration, extensional modes of vibration, bending modes of vibration, and flexural modes of vibration.

In one embodiment, the vibration element is configured so as to yield an extensional vibration coupled with a bending mode, both modes being excited by the extensional source. In this configuration, the bending mode is a harmonic of the extensional wave. This configuration yields an elliptical trajectory for each particle along the working edge of the probe. In this configuration, the equation of the curve for the booster portion of the motion profile is:

r=0.0625+0.002(e^{6.95x-6.45}−1),

0.5≦x≦1.0,

where r is the radius of the booster in inches, and x is the distance from the tip in inches.

In one embodiment of the invention, the vibrational element makes periodic transitions from a substantially compressed first state to a decompressed second state to a substantially stretched third state, while undergoing vibrational motion.

### BRIEF DESCRIPTION OF THE DRAWINGS

The invention can be more fully understood by referring to the following detailed description taken in conjunction with the accompanying drawings, in which:

FIG. 1 illustrates an overall schematic view of an ultrasonic surgical system, constructed in accordance with the present invention.

FIGS. 2A and 2B illustrate ultrasonic surgical instruments having vibration elements that are configured so as to enable vibration motion that includes a superposition of an extensional mode and a bending mode.

FIG. 3 illustrates an instantaneous longitudinal displacement profile for the surface of a vibration element depicted in FIGS. 3A and 3B, and determined by finite element analysis.

FIGS. 4A-4E illustrate a vibration element, which undergoes vibrational motion characterized by a periodic variation from a substantially compressed state (FIG. 4A) to an uncompressed state (FIG. 4B), then to a substantially stretched state (FIG. 4C), then back to the uncompressed state (FIG. 4D) and the stretched state (FIG. 4E).

FIG. 5 illustrates another embodiment of a vibration element, which shows a curved tip tuned for ultrasonic transmission.

### DETAILED DESCRIPTION

The present invention features a “multiple-wavelength” ultrasonic probe, having a vibrational element configured to support vibrational modes that are a superposition of a plurality of different modes of vibration, thereby enabling the simultaneous activation of multiple modes. In particular, the present invention is directed to intentional stimulation of vibrational motion that is perpendicular to the longitudinal axis of the ultrasonic probe. By stimulating transverse and/or rotational modes of vibration, the total vibration of the ultrasonic element is intentionally amplified.

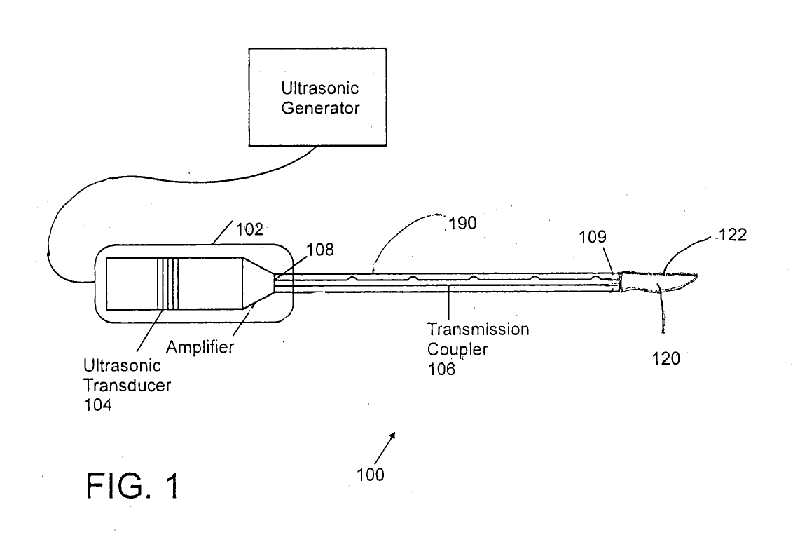

FIG. 1 illustrates an overall schematic view of an ultrasonic surgical system 100, constructed in accordance with the present invention. The system 100 includes at least one ultrasonic transducer 104. An ultrasonic generator is connected to the transducer 104, and supplies electric energy. The ultrasonic transducer 104 converts the supplied electric energy into ultrasonic frequency vibratory energy. The frequency range at which the system operates is typically between about 20 kHz and about 100 kHz, and the electric power supplied by the ultrasonic generator is typically between about 100 W to about 150 W. The ultrasonic transducer 104 may be made of piezoelectric material, or may be made of other materials, such as nickel, that are capable of converting electric energy into vibratory energy. The system may also include an amplifier (for example an acoustic horn), which amplifies the mechanical vibrations generated by the ultrasonic transducers.

The system includes an elongated ultrasonic transmission coupler 106 that extends along a coupler axis and has a proximal end 108 and a distal end 109. The ultrasonic coupler 106 is connected at the proximal end 108 to the transducer 104 to receive ultrasonic vibrations therefrom. The ultrasonic coupler 106 is adapted to transmit the ultrasonic vibrations received at the proximal end 108 to the distal end 109.

A vibration element 120 is connected to the distal end of the coupler, and receives ultrasonic vibrations from the coupler 106 so as to undergo vibrational motion. In an embodiment in which the vibration element 120 is used for cutting tissue, the vibration element 120 may be in the form of a blade, preferably having a blade edge 122 parallel to the coupler axis. In one embodiment of the invention, the vibration element is formed of a flexible, compliant material, for example a polymer. Examples of compliant materials that can be used to make the vibration element include, but are not limited to, polymer materials.

In one form of the invention, the vibration element has a substantially curvilinear configuration, for example curvilinear configurations disclosed in the AXYL-185PR application, referenced earlier.

In the present invention, the vibration element 120 is configured in such a way that the vibrational motion of the vibration element is a superposition of a plurality of vibratory modes. In a preferred embodiment of the invention, the vibration element 120 is configured so as to enable the simultaneous use of multiple modes of vibration to harmonically vibrate the vibration element 120. In one form, these multiple modes of vibration may all be excited by a single mode source. The individual constituent vibratory modes may include, but are not limited to, extensional modes of vibration, bending modes of vibration, flexural modes of vibration, transverse modes of vibration, and rotational modes of vibration.

Preferably, the vibration element 120 is configured so that the direction of the vibrational motion of the vibration element includes at least one component non-parallel to the coupler axis, i.e. the vibratory modes of the vibration element include non-longitudinal modes of vibration.

In a preferred embodiment of the invention, transverse and/or rotational modes of vibration are stimulated. In other words, the plurality of vibratory modes forming the composite mode of vibration of the vibration element includes 1) at least one transverse mode generated by a motion perpendicular to the longitudinal axis of the ultrasonic probe, and 2) at least one rotational mode generated by a rotational motion about the longitudinal axis.

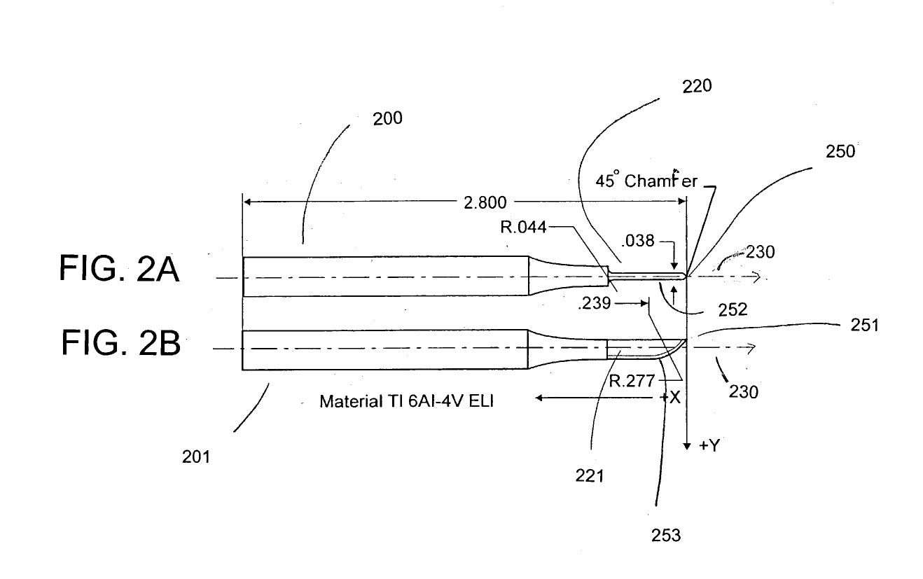

FIG. 2 illustrate ultrasonic surgical systems 200 and 201, which are constructed according to the preferred embodiment of the invention. In the illustrated embodiment, The vibration elements 220 and 221 are configured so as to amplify total vibration by stimulating transverse and/or rotational motion. In other words, motion of the vibrational element that is either perpendicular to the longitudinal axis (shown in FIG. 2 as 230) of the systems 200 and 201, or is rotational about the axis 230, is intentionally stimulated.

The configurations of the vibrational elements in FIG. 2 were designed to yield an extensional vibration, coupled with a bending mode. Both modes were excited by a single source, namely the extensional source. In the illustrated embodiment, the bending modes was not of the same wavelength as the extensional mode, but was a harmonic of the extensional mode. The design shown in the illustrated embodiments results from iterative methods, using finite element modal analysis. In other embodiments of the invention, the designs of the vibrational elements may be accomplished by trial and error, and by testing. As indicated in FIG. 2, the material from which the surgical systems 200 and 201 are fabricated is a titanium—aluminum alloy, more precisely Ti 6 Al—4V ELI.

The vibration elements 220 and 221 each include a tip 250 and 251, respectively. The vibration elements 220 and 221 also include at least one operative edge 252 and 253, respectively, along at least one side thereof. In the illustrated embodiment, the equation of the curve for the booster portion of the motion profile was:

r=0.0625+0.002(e^{6.95x-6.45}−1),

0.5≦x≦1.0,

where r is the radius of the booster in inches, and x is the distance from the tip in inches. The resulting trajectory for each particle along the operative edges 252 and 253 of the vibration elements 220 and 221 is an elliptical trajectory.

As seen from FIGS. 2A and 2B, the length of both the ultrasonic surgical systems 200 and 201, as measured from the proximal end 108 of the transmission coupler 106 to the distal tip of the vibration element, is about 2.800 inches. The vibration element 220 of the surgical system 200 has a booster radius of 0.044 inches, and a 45 degree chamfer at the distal tip of the vibration element. The width of the vibration element is 0.038 inches. The vibration element 221 of the surgical system 201 has a shape similar to a knife blade. The tapered portion of the vibration element 221 has a length of 0.239 inches. The booster radius of the surgical system 201 is 0.277 inches.

In the illustrated embodiments, transverse and/or rotational vibrational modes were stimulated, so as to develop a multi-dimensional velocity vector on the operative edge of the vibrational element. The resultant vector is time varying, and varies as a function of its position along the operative edge, to yield a time and position dependent velocity profile.

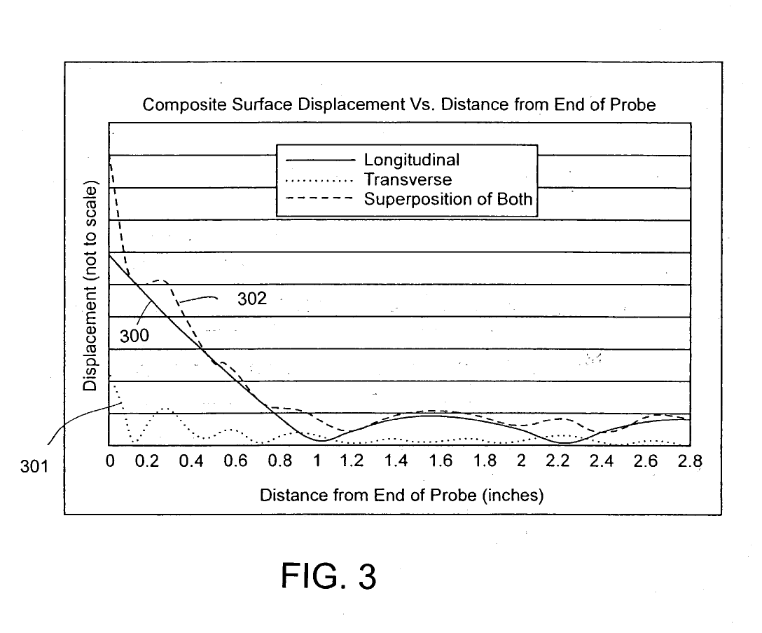

FIG. 3 illustrates velocity and displacement profiles for the surface of a exemplary vibration element that undergoes a vibrational motion consisting of a superposition of a extensional mode and a bending mode, as discussed in conjunction with FIG. 2. The curves shown in FIG. 3 were determined by finite element analysis, at a frequency of 75856 Hz.

The solid curve 300 shown in FIG. 3 illustrates the instantaneous longitudinal displacement profile, hence the velocity profile, of the surface of the vibration element depicted as 221 in FIG. 2. The instantaneous longitudinal displacement (not to scale) is shown as a function of the distance from the distal end of the probe, in inches. The instantaneous transverse displacement of the surface of the vibration element 221 is also shown, as a dotted curve 301, also as a function of the distance from the distal end of the probe. The superposition of 300 and 301, which is the resultant magnitude of the instantaneous displacement for the vibration element, is shown as a dashed curve 302, and is indicated in FIG. 3 as “Superposition of Both.” The resulting composite surface displacement curve (i.e. the dashed curve 302) is also shown as a function of the distance from the end of the probe. As discussed in conjunction with FIG. 2, the resulting trajectory for each particle along the working edge of the vibration element is an elliptical trajectory.

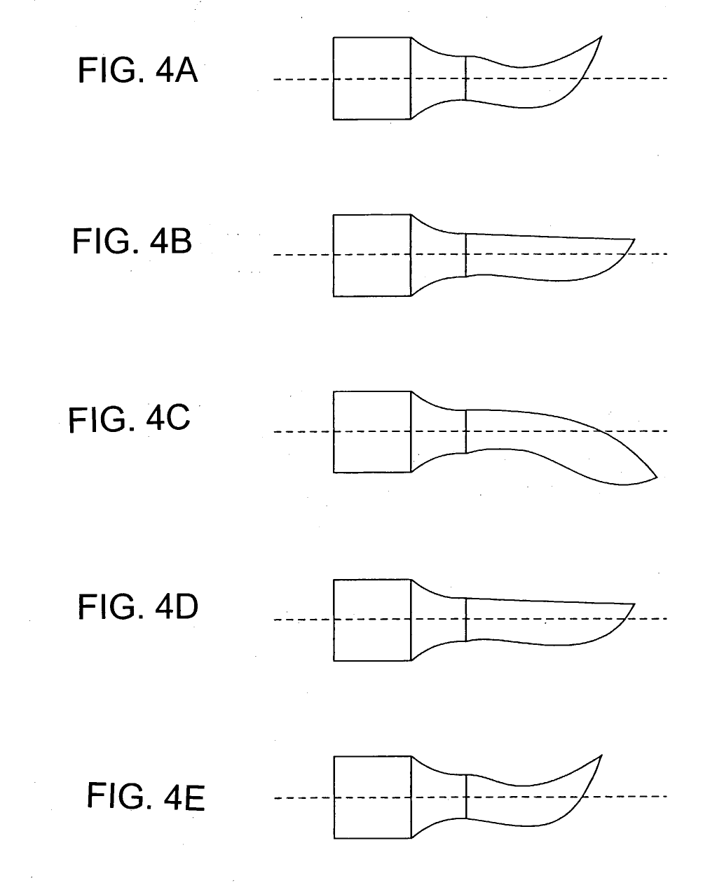

FIGS. 4A-4E illustrates another embodiment of the present invention, in which the vibrating element undergoes vibrational motion characterized by a periodic variation from a substantially compressed state to an uncompressed (or de-compressed) state to a substantially stretched state of the vibration element, upon receipt of ultrasonic vibrations transmitted through the coupler.

FIG. 4A illustrates the initial, substantially compressed state of the vibration element in the embodiment illustrated in FIGS. 4A-4E. FIG. 4B illustrates the subsequent de-compressed state of the vibration element. FIG. 4C illustrates the maximum stretched state of the vibration element. FIG. 4D illustrates the vibration element returning to an unstretched, and uncompressed state. FIG. 4E illustrates the final, substantially compressed state of the vibration element.

The modes of vibration illustrated in FIGS. 4A-4E may be formed, in one embodiment of the invention, by combining a longitudinal mode of vibration, with a torsional or twisting mode of vibration. Alternatively, the illustrated modes of vibration may be formed by combining a longitudinal mode of vibration with a flexural mode of vibration. Alternatively, the illustrated modes of vibration may be formed by combining a longitudinal mode of vibration with a rotational mode of vibration.

When the vibration element undergoes longitudinal modes of vibration, the vibration element moves back and forth along the longitudinal axis parallel to the coupler axis. By compounding the longitudinal modes with the torsional, flexural, or rotational modes, the vibration element undergoes the trajectory shown schematically in FIGS. 4A-4E as it moves from the substantially compressed state to the de-compressed stated to the substantially stretched state, then back to the substantially compressed state.

While the invention has been particularly shown and described with reference to specific preferred embodiments, it should be understood by those skilled in the art that various changes in form and detail may be made therein without departing from the spirit and scope of the invention as defined by the appended claims.

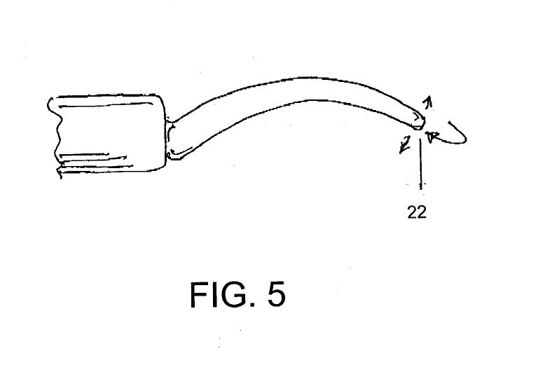

FIG. 5 illustrates another embodiment of a vibration element, which has a curved tip 22 tuned for ultrasonic transmission. Preferably, the curve is tuned to transmit maximal amplitude vibration at the tip 22.

What is claimed is:

1. An ultrasonic surgical instrument, comprising:
a. an ultrasonic transducer for generating ultrasonic vibrations;
b. an ultrasonic transmission coupler extending along a coupler axis and having a proximal end and a distal end, said ultrasonic coupler being connected at said proximal end to said transducer to receive ultrasonic vibrations therefrom, said ultrasonic coupler being adapted to transmit the ultrasonic vibrations received at said proximal end to said distal end; and
c. a surgical assembly connected to said distal end of said coupler, said surgical assembly including a;
wherein said vibration element is configured so that the direction of said vibrational motion of said vibration element includes at least one component non-parallel to said coupler axis.

2. An ultrasonic surgical instrument according to claim 1, wherein said vibrational motion of said vibration element comprises a superposition of a plurality of vibratory modes.

3. An ultrasonic surgical instrument according to claim 1, wherein said plurality of vibratory modes comprises at least one bending mode of vibration.

4. An ultrasonic surgical instrument according to claim 1, wherein said plurality of vibratory modes comprises at least one extensional mode of vibration.

5. An ultrasonic surgical instrument according to claim 1, wherein said vibration element is formed of a compliant material.

6. An ultrasonic surgical instrument according to claim 1, wherein said compliant material comprises polymeric material.

7. An ultrasonic surgical instrument according to claim 6, wherein said vibrational motion of said vibration element is characterized by a periodic variation in the state of said element from a substantially compressed first state to a substantially stretched second state.

8. An ultrasonic surgical instrument according to claim 1, wherein said vibration element is characterized by a substantially curvilinear configuration.

9. An ultrasonic surgical instrument, comprising:
a. an ultrasonic transducer for generating ultrasonic vibrations;
b. an ultrasonic coupler extending along a longitudinal axis, said coupler having a proximal end connected to said transducer to receive ultrasonic vibrations therefrom, said coupler being adapted to transmit the ultrasonic vibrations from said proximal end to a distal end of said coupler; and
c. a vibration element connected to said distal end of said coupler for receiving ultrasonic vibrations therefrom so as to undergo vibrational motion;
wherein said vibrational motion of said vibration element comprises a superposition of a plurality of vibratory modes; and
wherein said plurality of vibratory modes comprises at least one transverse mode that is generated by a motion perpendicular to said longitudinal axis.

10. An ultrasonic surgical instrument according to claim 9, wherein said plurality of vibratory modes comprises at least one extensional mode and at least one bending mode.

11. An ultrasonic surgical instrument according to claim 10, wherein said bending mode is a harmonic of said extensional mode.

12. An ultrasonic surgical instrument according to claim 11, wherein said vibration element comprises an operative edge, and wherein the trajectory undertaken by each particle along said operative edge as a result of said vibrational motion of said vibration element is substantially elliptical.

13. An ultrasonic surgical instrument according to claim 9, wherein said vibration element comprises an operative edge along one side thereof.

14. An ultrasonic surgical instrument according to claim 13, wherein said operative edge is characterized by a velocity profile generated as a result of said vibrational motion.

15. An ultrasonic surgical instrument according to claim 14, wherein said velocity profile is time dependent.

16. An ultrasonic surgical instrument according to claim 15, wherein said velocity profile is position dependent.

17. An ultrasonic surgical instrument according to claim 9, wherein said vibration element comprises a tip.

18. An ultrasonic surgical instrument according to claim 9, wherein said vibration element is characterized by a profile whose equation of curve for the booster portion is given by:
r=0.0625+0.002(e^{6.95x-6.45}−1),
0.5≦x≦1.0,
where r is the radius of the booster in inches, and
where x is the distance from said tip in inches.

19. An ultrasonic surgical instrument according to claim 9, wherein the configuration of said vibration element is developed using finite element modal analysis.

20. An ultrasonic surgical instrument, comprising:
a. an ultrasonic transducer for generating ultrasonic vibrations;
b. an ultrasonic coupler extending along a longitudinal axis, said coupler having a proximal end connected to said transducer to receive ultrasonic vibrations therefrom, said coupler being adapted to transmit the ultrasonic vibrations from said proximal end to a distal end of said coupler; and
c. a vibration element connected to said distal end of said coupler for receiving ultrasonic vibrations therefrom so as to undergo vibrational motion;
wherein said vibrational motion of said vibration element comprises a superposition of a plurality of vibratory modes; and
wherein said plurality of vibratory modes comprises at least one rotational mode that is generated by a rotational motion about said longitudinal axis.

21. An ultrasonic surgical instrument according to claim 6, wherein said vibrational motion of said vibration element is characterized by a periodic variation from a substantially compressed first state of said element to a de-compressed second state of said element to a substantially stretched third state of said element.
